(19) United States
(12) Patent Application Publication
(10) Pub. No.: US 2005/0119570 A1
(43) Pub. Date: Jun. 2, 2005

(54) Ultrasonic image and visualization aid

(76) Inventors: Stephen Lewis, Florissant, MO (US); James D. Taylor, Bridgeton, MO (US); Blake Ashby, University City, MO (US)

Correspondence Address: Paul M. Denk;Ste. 170, 763 S. New Ballas Road, St. Louis, MO, 63141, US

(21) Appl. No.: 11/001,371

(22) Filed: Dec. 1, 2004

Related U.S. Application Data
(60) Provisional application No. 60/526,046, filed on Dec. 1, 2003.

(51) Int. Cl. A61B 8/00

(52) U.S. Cl. 600/437

(57) ABSTRACT

Apparatus and method for providing ultrasound image and visualization aid that provides control and positioning of the ultrasonic technology and its scanning to furnish a more particular three dimensional graphical image and display during usage. The system provides for the display of an approximation of an organ or tissue mass being scanned, and the position of the image plane of the ultrasound relative to the determined approximation, to allow the ultrasound user to more quickly and accurately determine the location of the ultrasound scan plane in relation to the tissue mass.

### CROSS REFERENCE TO RELATED APPLICATION

This regular letters patent application claims priority to the provisional patent application having Ser. No. 60/526,046, which was filed on Dec. 1, 2003.

### FIELD OF THE INVENTION

This invention relates to the control and positioning of ultrasound technology and more particularly a three dimensional graphical image display for use with three dimesnional ultrasound systems. A further purpose of the invention is to display an approximation of an organ or tissue mass being scanned and the position of the scan plane of the ultrasound relative to the approximation of the tissue mass to allow an ultrasound user to more quickly and accurately understand the location of the ultrasound scan plane in relation to the tissue mass, improving the users ability to image the organ or tissues and guide treatments or surgical devices.

### BACKGROUND OF THE INVENTION

Ultrasound has become an important diagnostic tool for medical professionals. Generally, ultrasound scanning means can be categorized as either a “cavital” imaging device or a “body” imaging device. Cavital imaging devices, often referred to as “probes”, are often of a type that are inserted into a cavity in the patient to image organs within the cavity or juxtaposed to the cavity. Cavital probes are often specifically designed for the cavity to be imaged. Cavital probe types include trans rectal imaging probes, used for detection of prostate cancer and rectal cancer, and trans vaginal probes. Further, ultrasound is used for a variety of non-medical purposes as well, for example, checking mechanical parts for flaws or damage.

Ultrasound is inherently a two dimensional imaging modality, in that the image of an ultrasound system represents a very narrow slice of the imaged system. For this reason, it is difficult for ultrasound system users to initially interpret the position of the ultrasound scan plane in reference to the scanned tissue mass or organ. Consequently, users must spend time during an initial ultrasound scan moving the scan plane to survey the tissue mass or organ such that they understand the general location of the scan plane, or image being displayed, in reference to the tissue mass or organ.

Three Dimensional ultrasound imaging is an increasingly important diagnostic tool for physicians. Ultrasound system scanners are moved to capture multiple two dimensional scan planes in reference to a fixed point. The location of the scanner and scan plane can be captured in a number of ways. For a Cavital probe, this may include registers attached to a cradle in which a probe is affixed, external electromagnetic or optical sensors, which capture the specific location of the probe, or in the case of the Envisioneering Scanning Probe (U.S. Pat. No. 6,709,397), or a Solid State scanning probe, through the probe's internal control of the scan plane position. For a body imaging device, this may include registers attached to the device which work with external electromagnetic or optical sensors to capture the specific location of the body scanner. Three dimensional ultrasound is used to estimate the volume of organs, plan treatments and procedures, to guide less-invasive surgeries, and to guide targeted treatments. Three dimensional ultrasound is similarly important for non-medical uses, for example checking mechanical parts for flaws or damage.

A number of devices provide for the display of previously captured real images as comparison points. Further, a number of systems combine the images of different imaging modalities onto a single display.

Umemura (U.S. Pat. No. 4,598,368) discloses a device which will display and combine images from a plurality of imaging devices, such as X-Ray CT and NMR CT apparatus. All of the images to be displayed are “real”—an image of an actual tissue mass generated by an imaging means. P

Pelizzari, et al (U.S. Pat. No. 4,977,505) discloses a device which creates composite images from disparate sets of tomographic images, specifically for use with brain imaging. All of the images of the head and brain used in the composite image are “real”.

Hardy (U.S. Pat. No. 5,099,846) discloses a device for presenting a plurality of scanning images in a video presentation. The device displays previously captured images to allow their display side by side on a single video monitor.

Kenet, et al (U.S. Pat. No. 5,291,889) discloses a device for positioning a live image in reference to a previously stored image to allow a composite image to be displayed. Kenet utilizes a previously captured “real” image as its comparison point.

Schneider (U.S. Pat. No. 5,531,227) discloses a device for capturing in real time an image from one device which can be corresponded to the image and image point of view of a second device.

Gadonniex, et al (U.S. Pat. No. 5,538,003) discloses a means of allowing a user to superimpose a closed geographic figure over a previously scanned image, and then adjust the boundaries of the boundaries of the figure to more closely match an identified shape in the image.

Nafis, et al (U.S. Pat. No. 5,740,802), discloses a computer graphic and live video system which mixes images of the surface of a patient with computer generated models of internal organ. The computer generated models are derived from diagnostic images of the patient, i.e., previously captured “real” images.

Holupka, et al (U.S. Pat. No. 5,810,007) discloses a device for combining an ultrasound image with a CT image. The device utilizes an internally inserted ultrasound probe, which captures a close image of scanned tissue or an organ. Concurrently, an external CT image is taken, which shows the position of the probe in relation to the body. The device then inserts the ultrasound image from the probe into the CT image, to provide the greater level of detail.

Grimson, et al, (U.S. Pat. No. 5,999,840) discloses a system for capturing and displaying comparative three dimensional images. The images are captured by laser cameras, and then compared and combined on a video monitor.

Rottem (U.S. Pat. No. 6,032,678) discloses a device which is used as an adjunct to diagnostic imaging systems. The system uses a real time image with library stored images for assist doctors in making their diagnosis.

Hardy, et al (U.S. Pat. No. 6,240,308) discloses a device for archiving and simultaneously displaying brain scan images and maps.

Carol, et al (U.S. Pat. No. 6,325,758) discloses a method and apparatus for target position verification for radiation treatment. This method does include use of ultrasound images, however again all of the images used are “real”, captured from the patient.

However, all of these inventions suffer from a number of disadvantages. None allow the use of a non-real image, or approximation, for display. Further, none specifically address the goal of allowing a user to better understand the position of the image plane relative to a scanned tissue mass during an exam. Therefore, users would benefit from a display of a graphical image and current image plane.

### OBJECTS OF THE INVENTION

It is the principal object of this invention to provide a graphical image which, in conjunction with a projected image plane, allows the user of an imaging means to more quickly and accurately understand the location and position of the actual scan plane being generated by the imaging means, whether the imaging means is being used for medical or non-medical purposes.

Another object of the invention is to provide a graphical image which approximates the typical shape of a specific organ or tissue mass, or mechanical part.

Another object of the invention is to provide a graphical image which may be increased or decreased in size.

Another object of the invention is to provide a graphical image which may be increased or decreased in size in reference to data points selected by a user to with reference to a tissue mass or organ or mechanical part displayed on the imaging means display.

Another object of the invention is to provide a graphical image for which can be positioned using a single data point selected by the user to correlate to the boundary of an organ or mass being imaged by the imaging means.

Another object of the invention is to provide a graphical image for which multiple data points may be used to position the graphical image to correlate with the boundary of an organ or tissue mass or mechanical part as displayed on the imaging means display.

Another object of the invention is to provide the user with the approximate size and position of the organ or tissue mass or mechanical part within the probe's imaging volume.

These and other objects, advantages and features are accomplished according to the devices and methods of the following description of the preferred embodiment of the invention.

### BRIEF SUMMARY OF THE INVENTION

This invention relates primarily to a three dimesnional display technology that is able to generate a rendered three dimensional approximation of an organ or tissue mass or mechanical part being scanned, and the position of the scan plane of the ultrasound scanner relative to the approximation of the organ or tissue mass or mechanical part.

In reference to medical, the device consists of a series of saved graphical objects which are representative of tissue masses or organs of the human body, for instance a prostate or-rotator cuff of the shoulder, a scanning means with some form of scan plane position register, a means of noting the starting point and ending point of a scanned tissue mass or organ, means of proportionally scaling the saved graphical objects to correlate to the starting and ending points of the scanned tissue mass or organ, means of monitoring the horizontal and longitudinal location of the scan plane of an ultrasound system relative to a fixed point and means of displaying the horizontal and longitudinal location of the scan plane relative to the graphical object.

Referring to a scanning of the prostate, in use a trans-rectal ultrasound probe is placed in the cradle of a stabilizer. The user then advances and adjusts the cradle to allow the trans-rectal probe to be inserted into the rectum of a patient. The user generates an ultrasound image while positioning the probe to insure that the patient's prostate is viewable within the viewing area of the probe. If the user is using a scanning probe with the ability to move the probe plane without moving the probe, as disclosed in Envisioneering's Scanning Probe U.S. Pat. No. 6,709,397, or a Solid State/phased array scanning probe, with the probe imaging in Transverse mode the scanning probe is positioned such that the scan plane intersects the apex of the prostate, or the portion of the prostate most proximal to the use, and then locked into place. The user labels this scan plane position by pressing the “Apex” button on the ultrasound system. Next, with the probe still imaging in transverse mode, the user moves the scan plane until it intersects the base of the prostate, or the place most distal to the user. The user labels this scan plane position by pressing the “Base” button the ultrasound system.

From the library of stored graphical object, the user selects that object which equates with the shape of the prostate, or the user may select a default geometric shape, such as an ellipse. Based upon the Apex and Base landmarks identified previously, the stored graphical object is translated and scaled displayed on the monitor, appropriately placed within a wire frame representing the possible imaging volume of the probe. A semi-circular active imaging plane which correlates to the current position of the scan plane of the ultrasound system is superimposed over the graphical object, allowing the user to more easily identify the current position of the scan plane within the possible imaging volume and in reference to the organ or image mass being imaged. As the user changes the position of the scan plane of the ultrasound system, the active imaging plane indicator moves in reference to the stored graphical object, displaying the approximate location of the scan plane in reference to the tissue mass or organ being scanned.

### DRAWINGS

FIG. 1 discloses a perspective view of an ultrasound system utilizing the image plane visualization aid;

FIG. 2 discloses a side view of an ultrasound probe and stepper/stabilizer with external positioning registry;

FIG. 3 discloses a perspective view of an ultrasound body scanner with external positioning registry;

FIG. 4 discloses the indicator showing a transverse imaging plane approximately halfway between the base and apex planes in the center of the imaging volume;

FIG. 5 discloses the image plane visualization aid showing a transverse imaging plane that intersects the apex or most proximal point of the organ;

FIG. 6 discloses the image plane visualization aid showing a transverse imaging plane that intersects the base or most distal point of the organ; and

FIG. 7 discloses the image plane visualization aid showing a sagittal imaging plane approximately through the center of the organ.

### DRAWING NUMBERS

ultrasound system 1
cavital probe 2
cradle 3
stabilizer 4
ultrasound system CPU 5
cavital probe position register 6
monitor 7
probe tip 8
probe imaging window 9
Body Scanner 15
External Position Registers 16a, b & c
Graphical Object 20
Transverse imaging plane 21
Sagital active imaging plane 22
Possible Imaging Volume 23

### DESCRIPTION OF THE INVENTION

The following detailed description illustrates the invention by way of example and not by way of limitation. This description will clearly enable one skilled in the art to make and use the invention, and describes several embodiments, adaptations, variations, alternatives and uses of the invention, including what is presently believed to be the best mode of carrying out the invention. Additionally, it is to be understood that the invention is not limited in its application to the details of construction and the arrangements of components set forth in the following description or illustrated in the drawings. The invention is capable of other embodiments and of being practiced or being carried out in various ways. Also, it is to be understood that the phraseology and terminology used herein is for the purpose of description and should not be regarded as limiting.

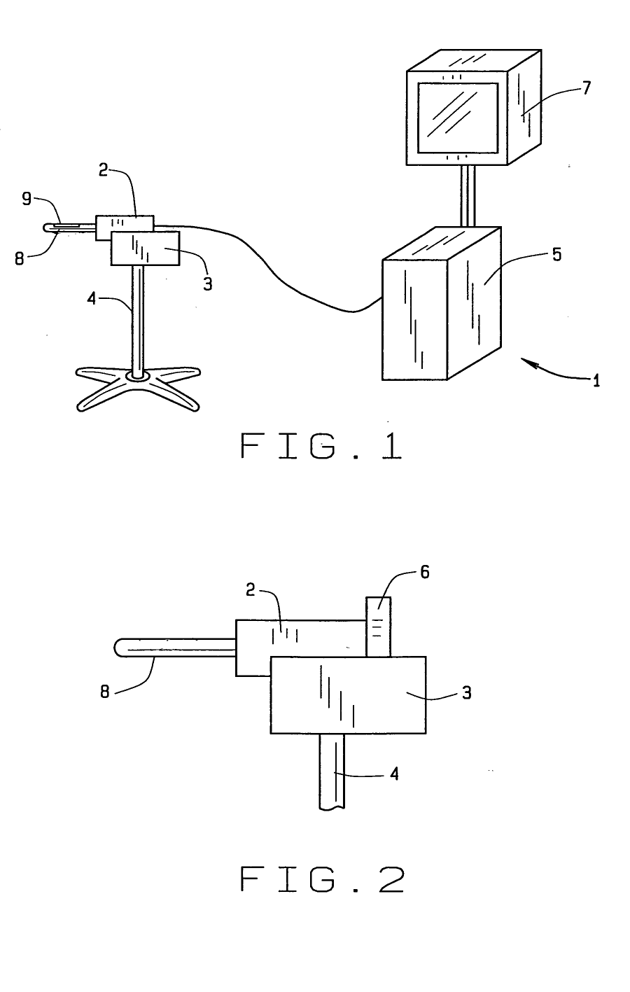

As seen in FIG. 1, the device consists of an ultrasound system 1, which in turn consists of a cavital probe, an ultrasound system CPU 5 and a monitor 7, a stabilizer 4 and a cradle 3. It is understood that the ultrasound system could be from a range of different manufacturers, for instance, manufactured by Siemens Medical Solutions, located in Malvern, Pa., or manufactured by Toshiba America Medical Systems, Inc., located in Tustin, Calif. As best seen in FIG. 4, the graphical representation consists of a graphical object 20, a transverse active imaging plane 21. FIG. 7 best displays the sagital image plane 22.

As seen in FIG. 2, a cavital probe 2 with cavital probe position register 6 may be used.

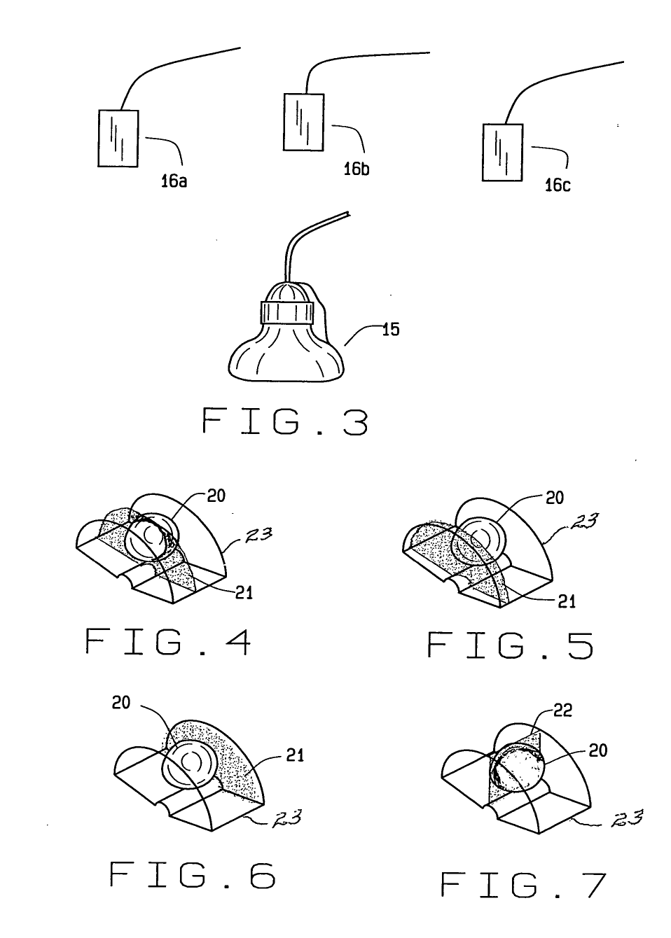

As seen in FIG. 3, a body scanner 15 with external position registers 16a, 16b and 16c may be used.

In operation, a cavital probe 2 is placed in the cradle 3 of a stabilizer 4. The user then advances and adjusts the cradle 4 to allow the cavital probe 2 to be inserted into the rectum of a patient. The user generates an ultrasound image while positioning the probe to insure that the patient's prostate is viewable within the probe imaging window 9 of the probe. If the user is using a scanning probe with the ability to move the probe scan plane without moving the probe, as disclosed in Envisioneering's Scanning Probe (U.S. Pat. No. 6,709,397), or a Solid State scanning probe with the probe imaging in Transverse mode the scanning probe is positioned such that the scan plane intersects the apex of the prostate, or the portion of the prostate most proximal to the user, and then locked into place. The user labels this plane by pressing the “Apex” button on the ultrasound system 1. Next, with the probe still imaging in transverse mode, the user moves the transverse scan plane until it intersects the base of the prostate, or the place most distal to the user. The user labels this plane position by pressing the “Base” button the ultrasound system 1.

Monitor 7 displays Possible Imaging Volume 23 showing a frame, such as a wire frame, representing the outer limits of the ultrasonic scan representing the possible imaging area.

From the library of stored graphical object, the user selects that graphical object 20 which equates with the shape of the prostate or the user may select a default geometric shape, such as an ellipse. Based upon the Apex and Base landmarks identified previously, the stored graphical object 20 is translated and scaled and displayed within the Possible Imaging Volume 23 wire frame, on the monitor 7. A semi-circular transverse active imaging plane 21 which correlates to the current position of the transverse scan plane of the ultrasound system 1 is superimposed over the graphical object 20, allowing the user to more easily identify the position of the scan plane within the imaging volume. The transverse active imaging plane 21 may partially obscure graphical object 20, and further the intersection of the transverse active imaging plane 21 and graphical object 20 may be highlighted on monitor 7. As the user changes the scan plane of the ultrasound system 1, the active imaging plane 21 moves in reference to the stored graphical object 20, displaying the approximate location of the image plane in reference to the scanned tissue mass or organ. The user may change to sagital imaging mode, in which the scan plane parallels the axis of the cavital probe. This causes monitor 7 to display sagital active imaging plane 22.

In an alternative embodiment as disclosed in FIG. 2, the device may be utilized with a traditional cavital probe 2 in conjunction with a cavital probe register 6. In use the cavital probe 2 is placed in the cradle 3 of a stabilizer 4. The user then advances and adjusts the cradle 4 to allow the cavital probe 2 to be inserted into the rectum of a patient. The user generates an ultrasound image while positioning the probe to insure that the patient's prostate is viewable within the probe imaging window 9 of the probe. With the probe imaging in transverse mode, the user positions the cavital probe such that the scan plane intersects the apex of the prostate. The user labels marks this cavital probe position in reference to the cavital probe register, labeling this position “Apex” on the ultrasound system 1. Next, with the probe still imaging in transverse mode, the user moves the cavital probe 2 in the cradle 3 until the scan plane intersects the base of the prostate. The user labels this plane by pressing the “Base” button the ultrasound system 1.

In an alternative embodiment as disclosed in FIG. 3, the device may be utilized with a traditional body scanner 15 in conjunction with external position registers 16a, 16b and 16c. The device may also be used with a body scanner utilizing Envisioneering's scanning technology as disclosed in Envisioneering's scanning probe patent (U.S. Pat. No. 6,709,397).

In an alternative embodiment, a single data point can be used to position the graphical object 20. The user sets one point as a reference, and then the device displays a static graphical object representing a static representation of an average organ. The organ size and position are determined by the program designer and cannot be adjusted by the user.

In a further alternative embodiment, more than two data points can be used. The device allows the user to label several planes or points on several images as specific landmarks. These landmarks allow the three dimensional display to adjust the size and placement of the representative organ within the displayed imaging volume. As the number of landmarks increases, the accuracy of the reconstruction improves. An approximately elliptical shaped organ like the prostate could be approximated with several landmark choices (in increasing order of position and size accuracy). Possible additional data points include two points indicating the widest transverse extent of the organ, and two points indicating the tallest extent of the organ or scanned mass. The image of the organ is moved and scaled so that its position and size approximate the organ position in the imaging volume.

In a further alternative embodiment, the graphical object's size and placement are determined by identification of the tissue boundaries of the organ. These boundaries can either be drawn by the user or can be determined automatically through a boundary recognition algorithm. The boundaries are used to first create a skeleton of the organ, and finally a surface rendering is made. The organ position and size are located within the imaging volume based on the positions of the boundaries.

As various changes could be made in the above constructions without departing from the scope of the invention, it is intended that all matter contained in the above description or shown in the accompanying drawings shall be interpreted as illustrative and not in a limiting sense. Further, while the above description addresses an ultrasound system utilized in medical imaging, it is understood that the device can be applied to non-medical imaging uses as well, for instance imaging mechanical parts.

1. An image plane visualization aid comprising:
a three dimensional graphical object;
a display;
an imaging means with a scan plane position registry;
such that the position of the scan plane of the imaging means can be approximated in reference to the graphical object.

2. The device of claim 1 whereby said imaging means is a cavital probe.

3. The device of claim 1 whereby said imaging means is a body scanner.

4. The device of claim 1 whereby a data point is used to position the edge of the graphical image with reference to the boundary of a tissue mass as displayed on the imaging means display.

5. The device of claim 1 whereby said graphical image may approximate the typical shape of a specific organ or tissue mass.

6. The device of claim 1 whereby the graphical image may be increased or decreased in size.

7. The device of claim 1 whereby the graphical image may be increased or decreased in size in reference to data points selected by a user to with reference to a tissue mass or organ displayed on the imaging means display.

8. The device of claim 1 whereby multiple data points are used position the graphical image to correlate with the boundary of a tissue mass as displayed on the imaging means display.

9. The device of claim 1 whereby said scan plane is a transverse scan plane.

10. The device of claim 1 whereby said scan plane is a sagital image plane.

11. The device of claim 1 whereby said graphical image is a rendered image generated from previously captured ultrasound images.

12. The device of claim 1 wherein the device is used for medical purposes.

13. The device of claim 1 wherein the device is used for non-medical purposes.
